(19) United States
(12) Patent Application Publication
(10) Pub. No.: US 2003/0144682 A1
(43) Pub. Date: Jul. 31, 2003

(54) Cardiac compression device, kit, and method of using same

(76) Inventors: Adnan I. Qureshi, Orange, NJ (US); Afshin A. Divani, Encino, CA (US)

Correspondence Address: CROWELL & MORING LLP, INTELLECTUAL PROPERTY GROUP, P.O. BOX 14300, WASHINGTON, DC, 20044-4300, US

(21) Appl. No.: 10/351,363

(22) Filed: Jan. 27, 2003

Related U.S. Application Data
(60) Provisional application No. 60/353,103, filed on Jan. 30, 2002.

(51) Int. Cl. A61M 29/00

(52) U.S. Cl. 606/191

(57) ABSTRACT

The cardiac compression device can comprise an esophageal insert having at least one magnet. The esophageal insert's magnet(s) may be attracted or repelled by one or more additional magnets located outside the esophagus. Controlled activation and/or deactivation of the additional magnet(s) causes the esophagus to move toward or away from a patient's heart, causing compression. A method of cardiac compression in a patient may be achieved by placing an esophageal insert comprising a magnet inside the esophagus of the patient and an excited external magnet, causing movement of the esophagus toward or away from the heart and thereby causing cardiac compression.

### FIELD OF THE INVENTION

The present invention relates generally to medical devices and methods. More specifically, the present invention relates to devices and methods useful in performing cardiac compression.

### BACKGROUND AND SUMMARY OF THE INTERVENTION

Insufficient cardiac output and sudden cardiac arrest are leading causes of morbidity and mortality in most modern societies. In patients exhibiting decreased cardiac output, temporary or permanent assistance in achieving optimum cardiac function is desired in order to provide a reasonably normal lifestyle. In cases of cardiac arrest, urgent reestablishment of cardiac function is required to prevent irreversible damage to viable organs, particularly the brain. By providing artificial circulation of oxygenated blood, relatively normal conditions can be maintained in the vital organs until normal heart function can be reestablished. See, inter alia, Davis and Nagel, “Complications of Cardiac Resuscitation” Chest 1987; 92:287-291.

In the case of cardiac failure, two types of cardiac compression techniques have been employed to apply pressure to the heart in order to maintain a sufficient amount of blood circulation. The first of these methods is external or closed (chest) cardiac massage, which consists of applying pressure on the anterior chest wall and alternatively releasing the pressure. When closed cardiac massage is combined with airway support, it is known as cardiopulmonary resuscitation (CPR).

CPR is a widely known procedure and may be employed by persons with basic skills and training. However, in order to be even minimally effective, the practitioner performing CPR must maintain chest compressions at an even distance of approximately 1.5 to 2 inches into the chest and at a rate of 80-100 compressions per minute. The compressions must be strong enough to sufficiently compress the chest cavity, but not too strong to prevent severe damage.

One drawback of this technique is that the applied pressure is not fully absorbed by the heart due to its location within the rib cage. In fact, the slight increase in cardiac output during CPR is generally attributable to the creation of negative pressure during chest compressions in the thorax with subsequent increase in venous return. The neurological and systemic morbidity during cardiac resuscitation is very high because poor amounts of oxygenated blood are provided to the organs with this limited cardiac output.

The second type of cardiac compression historically employed is internal or open (chest) cardiac massage. During open cardiac massage, the patient's chest is surgically opened and the heart is manually squeezed to pump blood throughout the body. This method provides desirable outcomes with regard to oxygenated blood flow. Obvious drawbacks exist, notably the requirement for a surgical facility and a team of highly trained professionals. When employed, the increased cardiac output must be balanced against the greater risk of injury, infection, and other related side effects of this invasive technique.

While there have been some advances in internal cardiac compression or massage, such as a minimally invasive incision through which fingers or small devices may be inserted, it still require advanced medical facilities and highly skilled care. There remains a need in the art for minimally invasive procedures which provide suitable cardiac output.

There also remains a need in the art for methods and devices which could be employed not only in cases of cardiac arrest, but which could also facilitate increased cardiac output in patients with, inter alia, coronary diseases, and who require temporary supplemental cardiac output assistance. Often these patients have been subjective to cumbersome pumps or extensive surgical procedures such as relocation and retraining of other bodily muscles around diseased or damaged portions of heart. It is therefore a goal of the present invention to provide improved devices and methods useful in cardiac compression. Such devices and methods could be used both to increase cardiac output in patients with various stages of cardiac insufficiency, and also to temporarily replace cardiac function in patients with cardiac failure.

According to one embodiment of the present invention, a cardiac compression device is provided which comprises an esophageal insert having at least one magnet. The esophageal insert could be in the form of a tube with a magnet and inflatable balloon. The magnet could be, for example, a plurality of magnetic strips.

According to a further embodiment of the present invention, a cardiac compression unit is provided which comprises an esophageal insert having at least one intraesophageal magnet, and at least one extraesophageal magnet. The extraesophageal magnet could be an electromagnetic device, and could also comprise a rechargeable battery or a DC generator supplying power to the electromagnetic device. The extraesophageal magnet could be placed outside a patient body, adjacent to the sternum and opposite the intraesophageal magnet. Alternatively or additionally, an extraesophageal magnet could be placed opposite a patient's posterior side of the rib cage. The invention could also include an electronic circuit capable of controlling the frequency, strength, and/or duration of attraction between the magnets provided therein.

According to a further embodiment of the present invention, a cardiac compression kit is provided, which comprises a cardiac compression unit and instructions for its use.

According to a further embodiment of the present invention, a method of cardiac compression in a patient is provided, comprising placing an esophageal insert comprising a permanent magnet or ferromagnetic material inside the esophagus of a patient in a location proximate to cardiac muscle, and placing a permanent magnet or an electromagnet against a patient's sternum opposite the esophageal insert, and/or by placing a magnet against a patient's posterior side of the rib cage facing the esophageal insert. Optimally, the method of cardiac compression can be performed at a rate of approximately 80-100 compressions per minute. However, the device is designed to operate with different duty cycles other than the aforementioned rate.

### BRIEF DESCRIPTION OF THE DRAWING FIGURES

FIG. 1 schematically represents a first embodiment of the device according to the present invention;

FIG. 2 shows a second embodiment of the device according to present invention;

FIG. 3 shows a third embodiment of the device according to the present invention;

FIG. 4 shows a sectional view from the side of a patient with a second embodiment of the unit according to the present invention;

FIG. 5 shows a cross sectional view of a patient with one embodiment of the unit according to the present invention;

FIG. 6 shows an embodiment of an extra-esophageal magnet according to the present intervention; and

FIG. 7 shows an embodiment of a kit according to the present invention.

### DETAILED DESCRIPTION OF THE DRAWING FIGURES

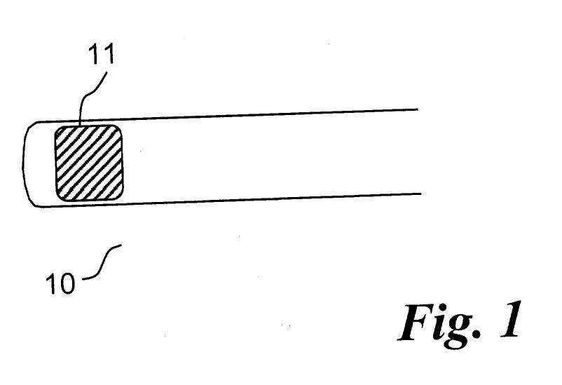

FIG. 1 shows a first embodiment of a device according to the present invention. Device 10 comprises a magnet 11, which could be a magnetized area or a separately-provided magnet(s). Device 10 is configured to be inserted in a patient's esophagus. Magnet 11 can be used in conjunction with a magnet external to a patient's esophagus in order to cause cardiac compression. Various methods for utilizing device 10 are explained in detail below.

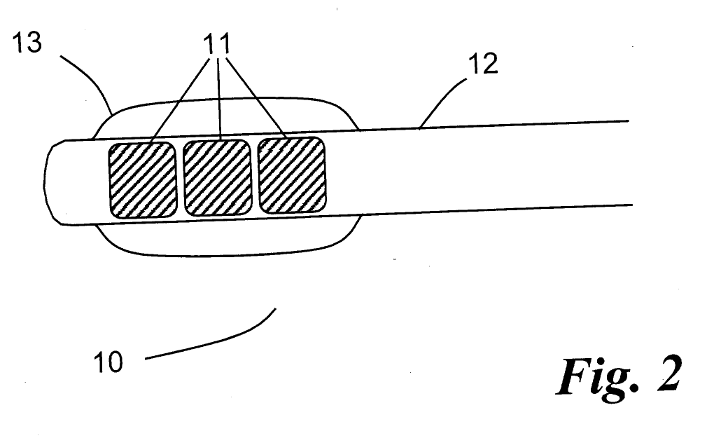

FIG. 2 shows a second embodiment of device 10 according to the present invention. Device 10 comprises a tube 12, an inflatable balloon 13, shown inflated, and magnetic strips 11. Magnet 11 is a plurality of magnetic strips in this embodiment. Tube 12 may facilitate manipulation of device 10, by having a shape and structure to provide ease of insertion and removal, and comfort and convenience during use.

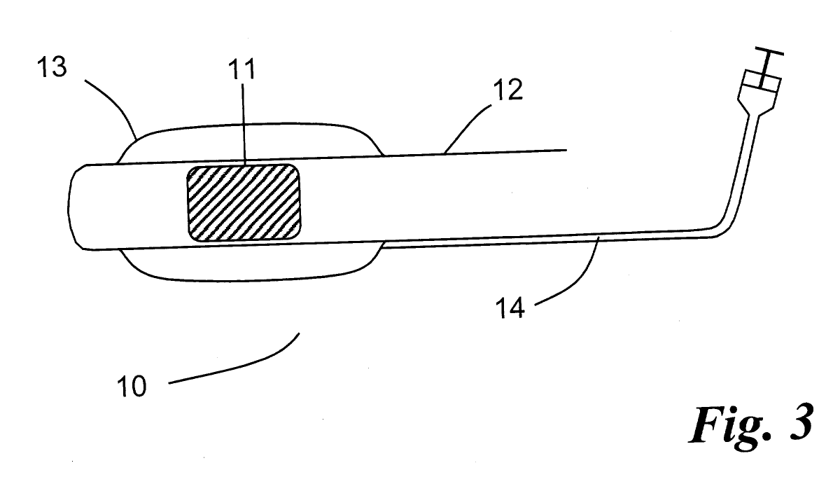

FIG. 3 shows a third embodiment of a device according to the present invention. Device 10 comprises a magnet 11, placed on tube 12. Tube 12 is locatable within a patient's esophagus (not shown). An inflatable balloon 13, shown inflated, is provided at a distal end of tube 12 in order to secure tube 12 inside the patient's esophagus. While distal positioning of balloon may be preferred, it is not required. Further, a balloon supply tube 14 is shown, wherein material such as fluid or air may be transported into or removed from balloon 13 in order to inflate or deflate balloon 13.

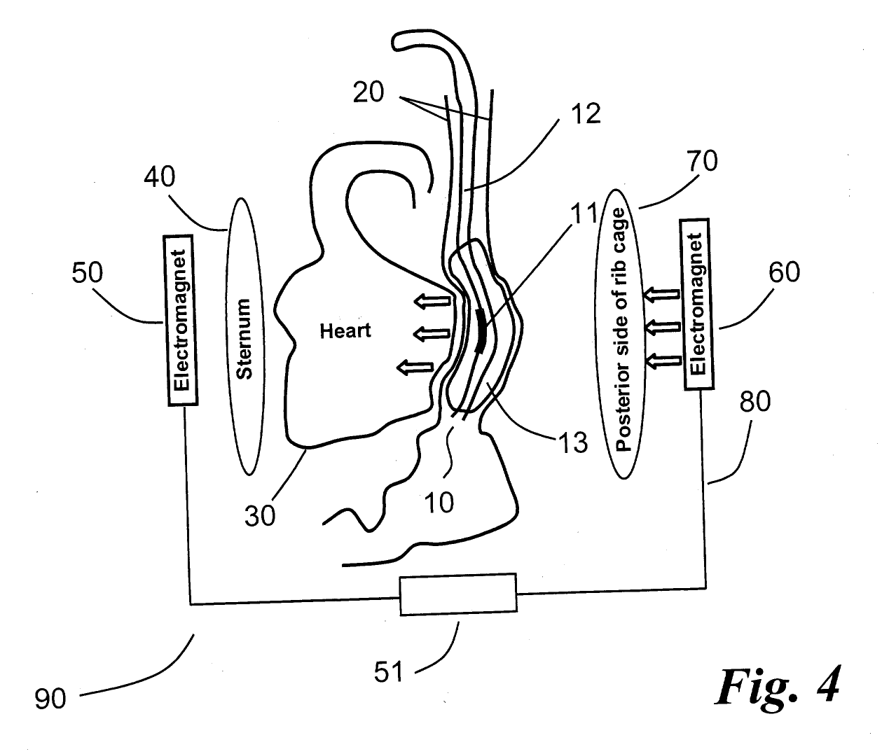

FIG. 4 schematically illustrates a sectional side view of a patient provided with a device 10 according to the present invention. Device 10 is shown in an esophagus 20. A heart 30 is shown, as well as a sternum 40. The device designated generally by the numeral 10 comprises a magnet 11 and a tube 12, and an inflatable balloon 13. Tube 12 may be inserted into esophagus 20, once positioned at the desired location, balloon 13 is inflated. Inflated balloon 13 holds device 10 in place with magnet 11 located adjacent to heart 30. A further or second magnet 50 may be provided, adjacent to sternum 40 and at a location opposite magnet 11 of device 10. In the event that magnet 50 is an electromagnet, a power source such as battery 51 is provided.

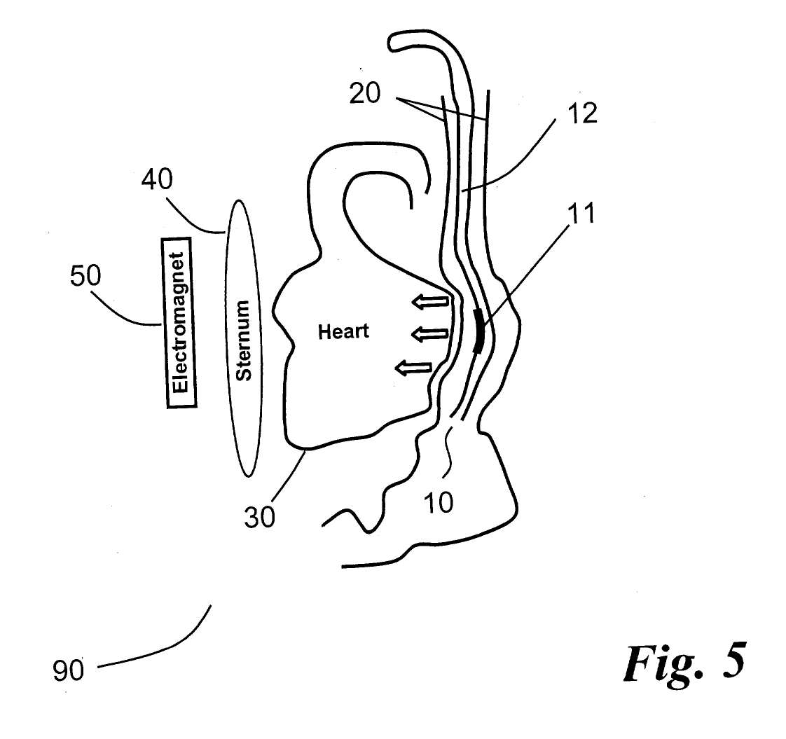

Magnet 50 may be external to the patient or subcutaneous. According to the embodiment depicted in FIG. 5, a third magnet 60 is provided, placed opposite the posterior side of ribcage 70 from device 10 and optionally connected to second magnet 50 with electrical connector 80. Electrical connector 80 may be particularly useful in coordinating a rapid but measured compression of heart 30. Where the patient suffers from inadequate cardiac function, subcutaneous magnets 50, 60 can be controlled using an external device using, for example, wireless communication technology. Switches (not shown) may be provided along electrical connector 80 according to known methods. A combination of device 10 and at least one additional magnet 50, 60 comprise a cardiac compression unit 90.

Multiple potential uses exist for the embodiment shown. For example, after insertion of device 10, magnet 50 may be provided with an opposite polarity to the magnet 11. The opposite magnetic fields are thus attracted to one another. Given the relative rigidity of sternum 40, magnet 50 cannot move toward magnet 11, but, given the relatively flexible nature of heart 30, magnet 11 can move toward magnet 50. This movement of magnet 11 toward magnet 50 causes compression of heart 30. The polarity of magnet 50 may optionally be reversed, causing magnet 50 and magnet 11 to have the same polarity. When that occurs, magnets 11 and 50 are repeled from one another, and if magnet 50 is held at a stable location adjacent to sternum 40, magnet 11 will travel away from magnet 50, allowing heart 30 to expand.

Alternatively or additionally, magnet 60 may be provided with a polarity opposite to magnet 11. The opposite magnetic forces are drawn toward one another and, given the relative rigidity of rib cage 70, magnet 60 is maintained in a relatively stable position while magnet 11 travels by being pulled toward magnet 60. This creates movement in esophagus 20 and provides negative pressure on heart 30, causing some blood flow within heart 30. Magnet 60 may then be reversed in polarity, causing magnet 60 to have the same polarity as magnet 11. The similarity in magnetic charge causes repulsion between magnets 60 and 11, wherein, if magnet 60 is held in a stable position, magnet 11 moves away from magnet 60, and toward heart. If sufficient repulsion exists, magnet 11 will travel toward heart 30 and return the esophagus 20 to its normal location, and continue traveling toward sternum 40, compressing heart 30 between esophagus 20 and sternum 40.

Electrical circuit 80 may be provided in order to control one or both of magnets 50, 60. This circuit could be configured within ordinary skill to control parameters such as frequency of alternation between magnet polarity, strength of magnet, and magnetization duty cycle. When properly employed, electrical circuit could help cardiac compression unit 90 reach optimum performance, which would be 80-100 compressions per minute of heart 30.

For convenience in depiction, the main intra-esophageal portion of the device is shown as a tube. The use of a tube has benefits. For example, the tube could be made hollow so as to form a second lining of patient's esophagus. This would allow normal swallowing function in patient when the device is in use. Alternatively, a closed tube could be provided and the tube could be filled partially or completely with air or fluid in order to make the device more rigid, or in order to expand the surface area of the esophagus. An expanded esophagus would create a greater surface area for compression of a patient's heart. It is within the scope of the present invention to have a one-piece device made of ferromagnetic material, which is selectively magnetized at a particular location which is opposite a patient's heart when the device is inserted. While an inflatable balloon facilitates use of the device, any other method to secure the device within a patient's esophagus may be used.

Using a device that provides a magnet or magnetic field inside a balloon allows the balloon to serve a dual function. It secures the position of the tube and magnet and it provides a cushion between the magnet and the esophagus. This may be preferred where the magnet could otherwise irritate or damage the esophagus during compression.

The types, sizes, and location of magnets used will be chosen based on ease of construction and practicality of use. For example, magnetic strips may be preferred because they can be placed adjacent to one another on a deflated balloon, and when the device is inserted into an esophagus and the balloon is inflated, the magnetic strips spread out and cover a greater surface area of the heart when it is compressed. The device can be built according to the present invention using readily available materials. For the portion of the device that is inserted into the esophagus, materials that are biologically compatible are contemplated. Further, materials that will suitably expand but not tear or damage internal organs should be employed. If the additional magnets are inserted, for example, subcutaneously, they should also be made using biocompatible materials. Care should also be taken so that the additional magnets, whether internal or external, cause minimal or no damage to a patient when used.

The internal magnet may preferably comprise rare earth magnets imbedded in the intra-esophageal tube. By embedding the magnets in the tube, the patient's tissue is not contacted by the magnet, which could be irritating. One material that may be used for the tube is medical grade silicone. External magnets may be formed from iron-core electromagnets. No direct contact between the external magnets and the patient is necessary. In the event that the internal or external magnets are positioned in immediate contact with a patient's tissue, the magnet(s) could be treated to prevent adverse interaction. For example, the magnet(s) could be coated with a biocompatible polymer.

FIG. 5 is a view from the side of a patient showing a cardiac compression unit 90 according to the present invention. As with all figures shown, shapes, sizes and positions are schematically represented only. While they depict relative positions of features to one another, the drawings are not extended to be anatomically correct or drawn to scale and are not critical to the scope of the present invention. In the embodiment of FIG. 5, a tube 12 is used as the intra-esophageal device, no balloon is provided. A single internal magnet 11 is provided to cause movement of esophagus 20 toward heart 30, thereby causing compression when attracted to second magnet 50.

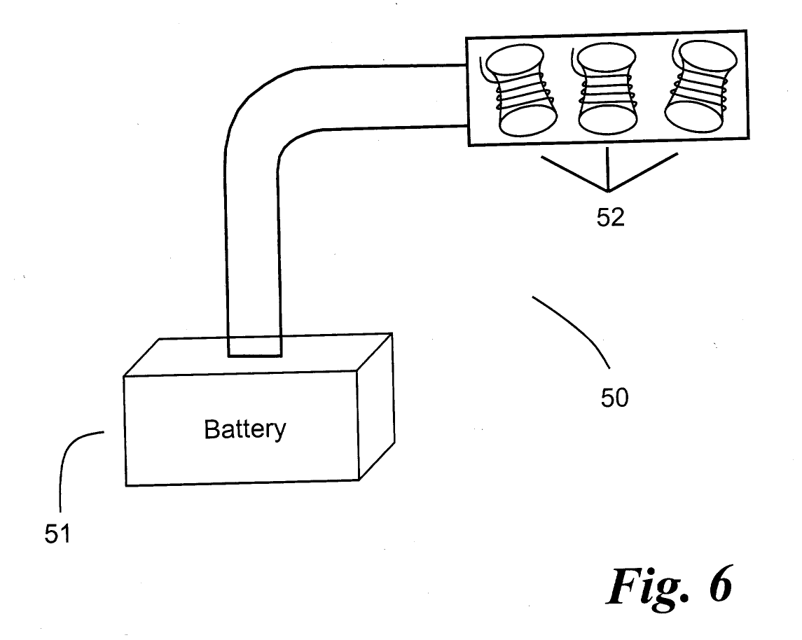

FIG. 6 shows another embodiment of a second magnet 50 according to the present invention, wherein magnet 50 comprises a battery 51 and a plurality of electromagnets 52. By exciting electromagnet(s) 52 via an electric circuit, the magnet 50 is activated and can be used to attract or repel a magnet located inside a patient. The direction of current flow dictates the polarity of the electromagnet(s) 52. The number and location of electromagnet(s) 52 can be varied as necessary in order to achieve the desired product size and magnetic capability. The battery 51 may be removably or fixedly connected to the electromagnet portion 52 of the magnet 50. The magnets in FIGS. 4 and 5 are shown schematically to represent any magnet, electromagnet, or ferromagnetic material. Electromagnets, for example, electromagnet 52 in FIG. 6, may be preferred for their precision and ease of manipulation. Where such devices are provided, a power source such as battery 51 of FIG. 6 is required. Power sources could include conventional batteries, rechargeable batteries, or other DC generators.

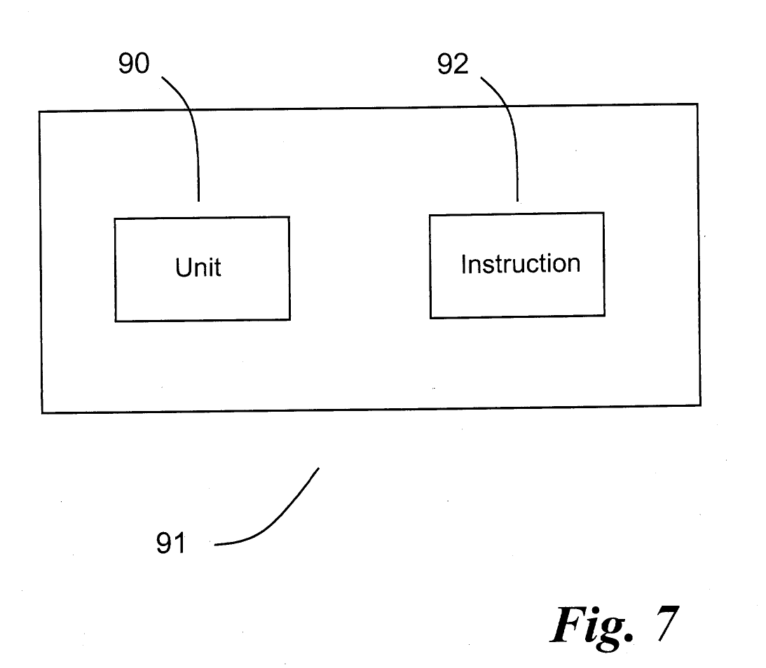

FIG. 7 schematically represents a kit 91 which includes the cardiac compression unit 90 according to the present invention, as well as instructions 92 for its use.

Although certain preferred embodiments and methods have been disclosed herein, it should be apparent for the foregoing disclosure to those skilled in the art that variations and modifications of such embodiments and methods may be made without departing from the spirit and scope of the invention. Therefore, the above description should not be taken as limiting the scope of the invention which is defined by the following claims.

We claim:

1. A cardiac compression device, comprising an esophageal insert having at least one magnet.

2. A cardiac compression device according to claim 1, wherein said esophageal insert is a tube.

3. A cardiac compression device according to claim 1, further comprising an inflatable balloon.

4. A cardiac compression device according to claim 1, wherein said at least one magnet comprises a plurality of magnetic strips.

5. A cardiac compression unit, comprising:
an esophageal insert with at least one intraesophageal magnet; and
at least one extraesophageal magnet.

6. A cardiac compression unit according to claim 5, wherein said extra-esophageal magnet comprises an electromagnetic device.

7. A cardiac compression according to claim 6, further comprising at least one of a rechargeable battery and a DC generator for excitation of said electromagnetic device.

8. A cardiac compression unit according to claim 5, wherein
said esophageal insert is configured to be placed inside the esophagus of a patient; and
said extraesophageal magnet is configured to be placed outside a patient body, adjacent to a sternum of said patient.

9. A cardiac compression unit according to claim 5, wherein
said esophageal insert is configured to be placed inside an esophagus of a patient; and
said extra esophageal magnet is configured to be placed outside a patient body, adjacent to a posterior side of a rib cage of said patient.

10. A cardiac compression unit according to claim 5, further comprising an electric circuit configured to control at least one of frequency, strength, and duration of attraction between said intra-esophageal magnet and said extraesophageal magnet.

11. A cardiac compression kit comprising:
a cardiac compression unit according to claim 5; and
instructions for use of said cardiac compression unit.

12. A method of cardiac compression in a patient, comprising:
placing an esophageal insert comprising a magnet inside an esophagus of the patient in a location proximate to cardiac muscle; and
exciting said magnet to cause cardiac compression.

13. A method of cardiac compression according to claim 12, further comprising:
placing a magnetic device against a sternum of the patient from said esophageal insert, wherein
said magnetic device performs said exciting step.

14. A method of cardiac compression according to claim 12, further comprising:
placing a magnetic device against a posterior side of a ribcage of the patient from said esophageal insert, wherein
said magnetic device performs said exciting step.

15. A method of cardiac compression according to claim 12, wherein said the compression is performed at an adjustable rate of approximately 80-100 compressions per minute.
